(19) United States
(12) Patent Application Publication
(10) Pub. No.: US 2008/0261869 A1
(43) Pub. Date: Oct. 23, 2008

(54) COMPOSITIONS AND METHODS OF USE FOR ALPHA-1 ANTITRYPSIN HAVING NO SIGNIFICANT SERINE PROTEASE INHIBITOR ACTIVITY

(76) Inventor: Leland Shapiro, Denver, CO (US)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 12/106,052

(22) Filed: Apr. 18, 2008

Related U.S. Application Data
(60) Provisional application No. 60/913,174, filed on Apr. 20, 2007.

(51) Int. Cl. A61K 38/17; C07K 14/435; C07K 1/00; A61P 1/16; A61P 9/00; A61P 11/00; A61P 31/12; A61P 31/04; A61P 31/18; A61P 37/00

(52) U.S. Cl. 514/8; 530/395; 530/402

(57) ABSTRACT

Embodiments herein illustrate methods and compositions for treating medical disorders. In certain embodiments, compositions and methods relate to reducing, inhibiting or treating a bacterial infection, or a viral infection in a subject. More particularly, embodiments herein relate to compounds including naturally occurring and synthetic compositions having alpha-1 antitrypsin activity but no significant serine protease inhibitor activity.

### PRIORITY

This application claims the benefit under 35 USC § 119(e) of provisional U.S. patent application Ser. No. 60/913,174 filed on Apr. 20, 2007, which is incorporated herein by reference in its entirety.

### FIELD

Embodiments herein relate to compositions, methods and uses for alpha-1 antitrypsin (α-1 antitrypsin, AAT) or analog thereof having no significant serine protease activity. In certain embodiments, α-1 antitrypsin can have significantly reduced or eliminated serine protease activity. Other embodiments relate to compositions and methods for treatment of medical conditions associated with viral infections, bacterial infections, apoptosis-mediated conditions and cytokine-mediated conditions.

### BACKGROUND

Normal plasma concentration of alpha-1 antitrypsin (AAT) ranges from 1.3 to 3.5 mg/ml. Under certain conditions, AAT can behave as an acute phase reactant and increase 3-4-fold during host response to inflammation and/or tissue injury or dramatic change such as with pregnancy, acute infection, and tumors. AAT easily diffuses into tissue spaces and forms a 1:1 complex with target proteases, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen. Therapeutic AAT has been commercially available since the mid 1980's and is prepared by various purification methods. Prolastin is a trademark for a purified variant of AAT. Recombinant unmodified and mutant variants of AAT produced by genetic engineering methods are available.

### Human Immunodeficiency Virus (HIV)

Previous research has shown that replication of FHV requires protease activity amongst other activities for the cleavage of gag-pol precursor proteins. This enzymatic activity is similar to activity of renin-aspartyl protease produced by the kidney. The close relationship between renin and HIV encoded protease led to an accelerated generation of specific HIV protease inhibitors as effective agents in treatment of AIDS. Many therapeutic agents directed against HIV protease have been developed as a consequence and used successfully in AIDS patients. For example, indinavir and crixivan are aspartyl protease inhibitors, which inhibit cleavage of pre-protein of HIV by viral own protease and thereby suppress viral proliferation. These agents have had some success but more rigorous treatments for HIV are needed.

### Influenza Virus

Influenza is an orthomyxovirus. Three genera, types A, B, and C of influenza exist. Types A and B are the most clinically significant, causing mild to severe respiratory illness. Influenza B is a human virus and does not appear to be present in an animal reservoir. Type A viruses exist in both human and animal populations, with significant avian and swine reservoirs. Although relatively uncommon, it is possible for nonhuman influenza A strains to infect humans by jumping from their natural host. In one specific example, the highly lethal Hong Kong avian influenza outbreak in humans in 1997 was due to an influenza A strain H5N1 virus that was an epidemic in the local poultry population at that time. In this case, the virus killed six of the 18 patients shown to have been infected.

Annual influenza A virus infections have a significant impact on humanity both in terms of death, between 500,000 and 1,000,000 worldwide each year. In addition, economic impact is huge resulting from direct and indirect loss of productivity during infection. One of the most dramatic events in influenza history was the so-called “Spanish Flu” pandemic of 1918-1919. In less than a year, between 20 and 40 million people died from influenza, with an estimated one fifth of the world's population infected. The virus that caused the Spanish flu was unique for several reasons, not the least of which was its ability to kill previously healthy young adults. In fact, the US military was devastated by influenza near the end of World War I, with 80% of US army deaths between 1918 and 1919 due to infection. Because it is a readily transmitted, primarily airborne pathogen, influenza A represents a serious concern.

### TB and MAC

Mycobacterium is a genus of bacteria which are aerobic, mostly slow growing, slightly curved or straight rods, sometimes branching and filamentous, and distinguished by acid-fast staining. Typically, mycobacteria are gram-positive obligate aerobes. The genus mycobacterium includes the highly pathogenic organisms that cause tuberculosis (M. tuberculosis and sometimes M. bovis) and leprosy (M. leprae). There are, however, many other species of mycobacterium.

Certain mycobacteria other than M. tuberculosis and M. bovis are alternatively known as non-tuberculosis mycobacteria. They are divided into four groups, also known as Runyon groups, based on pigmentation and growth rate. Each group includes several species. Group I refers to slow-growing photochromogens; Group II refers to slow-growing scotochromogens; Group III refers to slow-growing nonphotochromogens; and Group IV refers to rapidly-growing mycobacteria. The non-tuberculosis mycobacteria are also called atypical or anonymous mycobacteria.

Tuberculosis is an acute or chronic infectious disease caused by infection with M. tuberculosis. Tuberculosis is a major disease in developing countries, as well as an increasing problem in developed areas of the world, with approximately 8 million new cases and 3 million deaths each year. Although the infection may be asymptomatic for a considerable period of time, the disease is most commonly manifested as an acute inflammation of the lungs, resulting in fever and a nonproductive cough. If left untreated, serious complications and death typically result.

Although tuberculosis may be controlled using extended antibiotic therapy for an infected individual, such treatment is not sufficient to prevent the spread of the disease. Treatment regimens often require six to twelve months of uniterrurpted therapy. As a result, many patients do not complete the course of treatment, thus leading to ineffective treatment and development of antibiotic resistance TB. Effective vaccination and accurate, early diagnosis of the disease are needed in order to inhibit the spread of tuberculosis. Vaccination with live bacteria remains the most efficient method for inducing protective immunity. The most common Mycobacterium employed in the live vaccine is Bacillus Calmette-Guerin (BCG), an avirulent strain of Mycobacterium bovis. Some countries, such as the United States, however, do not vaccinate the general public because of concerns regarding the safety and efficacy of BCG. Thus, a need exists for alternative treatments to prevent the spread of TB and more rapidly treat an infected individual.

### Mycobacterium Avium Complex (MAC)

MAC infections currently account for approximately 50% of the pathogenic isolates identified by mycobacteriology labs and are most common among AIDS and other immuno-compromised patients. Early diagnosis and treatment of MAC infections can improve and prolong the lives of infected individuals.

### Anthrax and Anthrax Toxin

Anthrax toxin, produced by the gram positive rod-shaped aerobic, spore-forming bacterium Bacillus anthracis, is the toxic virulence factor secreted by this organism. B. anthraxis is often considered for use as a biological weapon due to the potency of the secreted exotoxin, and to the capacity of the bacterium to form dormant spores which resist harsh environmental conditions. Sporulation enables ready transport and distribution of large quantities of toxin-producing bacteria.

Because of some of the difficulties and inadequacies of conventional therapy for tuberculosis, other mycobacterial infections, and anthrax, new therapeutic modalities are desirable.

This invention thus addresses a need for safe and effective methods of treatment of tuberculosis, other mycobacterial infections, other Gram negative and Gram positive bacterial infections, viral infections, apoptosis-mediated diseases and cytokine mediated diseases.

### SUMMARY

Embodiments herein provide for methods and compositions for treating a subject having a medical disorder. Other embodiments provide for methods and compositions for treating a subject exposed to a microorganism such as a virus or bacteria. In accordance with these embodiments, a disorder may include, but is not limited to, a viral infection disorder, a bacterial infection disorder or a combination thereof.

Certain embodiments concern compositions for treating a subject having a medical disorder. In accordance with these embodiments, a composition can include, alpha-1 antitrypsin, or alleles thereof (for example, there are approximately 100 naturally occurring AAT varients), or fragments thereof or analogs thereof or fusion protein thereof (e.g. a human IgG or fragment of human IgG) where either composition has no significant serine protease inhibition activity. In further embodiments, a composition contemplated herein includes, but is not limited to, modifying the composition to increase stability of the composition (e.g. polyethylene glycol linked molecules such as AAT or fragment thereof having no significant serine protease inhibition activity, etc.) It is contemplated herein that a composition may include a deglycosylated form of AAT or fragment thereof, analogs thereof, or recombinant form thereof, having no significant serine protease inhibition activity. Some embodiments herein include, but are not limited to, a composition using AAT having no significant serine protease inhibition activity wherein the AAT is naturally occurring M phenotype. In other embodiments, a composition may further include, but is not limited to, an anti-inflammatory agent, an immunosuppressive agent, an immunomodulatory agent, an anti-microbial agent, an anti-viral agent, an anti-bacterial agent, or combinations thereof.

In certain embodiments, a viral infection can be a retroviral infection. In more particular embodiments a retroviral infection can include but is not limited to, HIV infection, AIDS (acquired immunodeficiency syndrome), influenza virus infection, hepatitis virus infection, Herpes virus infection and a combination thereof.

In other embodiments, a bacterial infection contemplated herein can include, but is not limited to, mycobacterial infection, sepsis, septic shock, bacterial meningitis, bacterial pneumonia, and anthrax disease. In certain embodiments, anthrax disease can be derived from the group consisting of inhalation anthrax, cutaneous anthrax, gastrointestinal anthrax or combinations thereof.

Compositions contemplated herein may further include an agent selected from the group consisting of an anti-inflammatory agent, an immunosuppressive agent, an immunomodulatory agent, an anti-viral agent, an anti-pathogenic agent, an anti-bacterial agent, a reverse transcriptase inhibitor, a protease inhibitor, and a combination thereof.

In certain embodiments, compositions herein can be administered orally, systemically, via an implant, time released or slow-release compositions (e.g. gel, microparticles etc.), intravenously, topically, intrathecally, subcutaneously, by inhalation, nasally, or by other means known in the art or a combination thereof.

Methods of treatment contemplated herein can further include reducing or eliminating one or more symptom associated with a medical disorder in a subject including, but not limited to, ulceration, scar formation, pulmonary edema, peripheral edema, hemorrhage, necrotizing mediastinal, lymphonaphy, plueral effusion, ventilatory compromise, cough, sweating, rigors, malaise, fever, dry cough, myalgias, chest pain, cutaneous ulceration, edema, non-pitting edema, escar, nausea, diarrhea, vomiting, abdominal pain, combinations thereof, or preventing or reducing the risk of death of the subject.

Certain methods of treatment further concern reducing or eliminating one or more symptoms associated with a medical disorder. Further, some embodiments herein include symptoms that are characteristic of a disease, infection or onset thereof.

In a more particular embodiment, a viral medical disorder can include an influenza infection. In accordance with these embodiments, the influenza infection can include influenza A or B infection.

In other embodiments, a composition can further include one or more anti-inflammatory agents, immunosuppressive agents, immunomodulatory agents, anti-microbial agents, anti-viral agents or a combination thereof.

Some embodiments herein concern compositions of use for reducing or eliminating serine protease inhibition activity in the composition where no significant serine protease inhibitor activity is detectible. In accordance with these embodiments, alpha-1 antitrypsin, a fragment thereof, an analog thereof, alleles thereof or fusion molecule thereof, or combinations thereof can be heated to a temperature of about 85° C. to about 100° C. for about 1 minute to about 40 minutes, or about 5 minutes, or 10 minutes etc. In other particular embodiments, alpha-1 antitrypsin, a fragment thereof, an analog thereof, or fusion molecule thereof, can be heated and/or chemically treated until no significant serine protease inhibitor activity is detected. Certain methods can further include assessing serine protease inhibition activity of the alpha-1 antitrypsin, a fragment thereof, an analog thereof, or fusion molecule thereof, or combinations thereof using a serine protease inhibitor activity assay. It is contemplated herein that serine protease inhibitor activity can be measured before and/or after treatment.

In other embodiments, compositions disclosed herein may further contain an agent including, but not limited to, an anti-inflammatory agent, an immunosuppressive agent, an immunomodulatory agent, an anti-microbial agent, an anti-viral agent, an anti-bacterial agent, an anti-fungal agent and combinations thereof.

In certain embodiments, treating, ameliorating symptoms from and preventing bacterial diseases is provided. Embodiments herein can relate to compositions and methods for inhibition of Gram negative, Gram positive and acid fast bacilli. In accordance with these embodiments, certain bacteria are contemplated herein, for example, tuberculosis (TB), mycobacterium avium complex (MAC), and anthrax. It is contemplated herein that compositions and methods can include, but are not limited to modulation of cellular activities such as macrophage activity or induced inflammation caused by immune responses from bacterial infections.

In some embodiments, bacterial infections that may be treated or ameliorated using compositions and methods disclosed herein are those infections caused by Gram negative bacterial organisms including, but not limited to, N. gonorrhoeae, N. meningitidis, M. catarrhalis, H. influenzae, E. coli, all Klebsiela spp., all Enterobacter spp., all Serratia spp., all Salmonella spp., all Shigella spp., Proteus mirabilis, Proteus vulgaris, all Providencia spp., all Morganella spp., all Citrobacter spp., all Aeromonas spp., all Acinetobacter spp., Pseudomonas aeruginosa, all Pasteurella spp., Pseudomonas cepacia, Stenotrophomonas maltophilia, Y. enterocolitica and other Yersinoiiosis, all Legionella spp., P. multocida, H. ducreyeii, all Chlamyidia spp., Mycoplasma pneumoniae, Mycoplasma hominis, Bacteroides fragilis, P. melaminogenica, all Moraxella spp., all Bortedella spp., or any combination thereof.

In another embodiment, bacterial infections that may be treated or ameliorated using the compositions and methods of the invention are those infections caused by Gram positive bacterial organisms including, but not limited to, C. tetani, C. botulinum, C. difficile, Group A, B C, and G Streptococcus, Streptococcus pneumoniae, Streptococcus milleri group, Viridans streptococcus, all Listeria spp., all Staphylococcus spp., S. aureus (MSSA), S. aureus (MRSA), S. epidermidis, Enterococcus faecalis, Enterococcus faecium, all Clostridium spp. including C. diptheriea, C. jeikium, all Rhodococcus spp., all Leukonostoc spp. or any combination thereof.

In certain embodiments, the mycobacterium inhibited from infecting macrophages in a mammal in need thereof including, but not limited to, a mycobacterium such as a non-tuberculosis mycobacteria from four Runyon groups including Group I (slow-growing photochromogens), Group II (slow-growing scotochromogens), Group III (slow-growing nonphotochromogens), and Group IV (rapidly-growing mycobacteria).

In yet other embodiments, infections that may be treated, eliminated or ameliorated using the compositions and methods contemplated herein are those infections caused by mycobacterium, including, but not limited to, highly pathogenic organisms that cause tuberculosis (M. tuberculosis and sometimes M. bovis) and leprosy (M. leprae). There are, however, many other species of mycobacterium such as M. avium-intracellulare, M. chelonei (also known as borstelense and abscessus), M. africanum, M. marinium (also known as balnei and platypoecilus), M. buruli (also known as ulcerans), M. fortuitum (also known as giae, minetti, and ranae), M. haemophilum, M. intracellulare, M. kansasii (also known as luciflavum), M. littorale (also known as xenopi), M. malmoense, M. marianum (also known as scrofulaceum and paraffinicum), M. simiae, M. szulgai, M. ulcerans, or any combination thereof are also contemplated.

In certain embodiments, compositions and methods disclosed herein can be used to reduce or prevent pain and/or symptoms associated with medical indications. In accordance with these embodiments, reduction in pain and or symptoms associated with a medical indication is on the order of about 10-20%, or about 30-40%, or about 50-60%, or about 75-100% reduction or inhibition.

In certain particular embodiments, symptoms associated with anthrax disease that can be reduced or prevented include, but are not limited to, malaise, fever, dry cough, myalgias, and chest pains, ventilatory compromise, sweating, widening of the mediastimum on radiographic studies, edema of the neck and chest, necrotizing mediastinal lymphadenitis, non-pitting edema, eschar, nausea, vomiting, fever, abdominal pain, bloody diarrhea, mucosal ulcerations, hemorrhagic mesenteric lymphadenitis, any combination thereof, or death caused by bacillus anthracis exposure or infection.

Other embodiments concern a method of relieving or ameliorating the pain or symptoms associated with any one or more of the above-identified bacterial diseases or indications, mycobacterial diseases or indications, or anthrax infection in a mammal suffering from any one or more of the above-identified conditions which can include administering to a subject in need of a treatment a therapeutically effective amount of a composition having no significant serine protease activity. For example, a composition can include alpha-1 antitrypsin, a fragment thereof, an analog thereof, alleles thereof or fusion molecule thereof, wherein the molecule is treated to reduce or eliminate serine protease inhibitor activity where no significant amount of serine protease inhibitor activity remains. In accordance with these methods, the treatment is sufficient to inhibit or ameliorate the bacterial disease or indication, mycobacterial disease or indication, or anthrax infection of the host.

In certain embodiments, α1-antitrypsin used in the methods and compositions herein can include, but is not limited to, Aralast™ (Baxter), Zemaira™ (Aventis Behring), Prolastin™ (Bayer), Aprotonin™ or Trasylol™ (Bayer Pharmaceutical Corporation) and Ulinistatin™ (Ono Pharmaceuticals, Inc.) or any combination thereof. In other embodiments, AAT or an AAT fragment or an AAT analog used in methods and compositions herein can include naturally occurring AAT or AAT fragment or analog or allele thereof.

In other embodiments, an anti-inflammatory compound or immunomodulatory drug can include, but is not limited to, interferon; interferon derivatives comprising betaseron, β-interferon; prostane derivatives comprising iloprost, cicaprost; glucocorticoids comprising cortisol, prednisolone, methylprednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof, soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in any combination thereof.

In certain embodiments, compositions for administration can be in a range of between about 10 ng and about 10 mg per ml or mg of the formulation. A therapeutically effective amount of AAT peptides or drugs that have similar activities as AAT or peptides drug may be measured in molar concentrations and may range between about 1 nM and about 10 mM. The formulation is also contemplated in combination with a pharmaceutically or cosmetically acceptable carrier. Precise doses can be established by well known routine clinical trials without undue experimentation.

In some embodiments, pharmaceutical compositions contemplated herein are administered orally, systemically, via an implant, intravenously, topically, intrathecally, intracranially, intraventricularly, by inhalation or nasally.

In certain embodiments, the subject or mammal is a human.

In other embodiments, the subject or mammal can be a domesticated or a non-domesticated mammal.

In certain embodiments, synthetic and/or naturally occurring peptides may be used in compositions and methods herein for example, providing other than serine protease inhibitor activity of AAT. Homologues, natural peptides, with sequence homologies to AAT including peptides directly derived from cleavage of AAT may be used or other peptides such as, peptides that have AAT-like activity other than serine protease inhibitor activity. Other peptidyl derivatives, e.g., aldehyde or ketone derivatives of such peptides are also contemplated herein. Without limiting to AAT and peptide derivatives of AAT, compounds like oxadiazole, thiadiazole and triazole peptoids and substances can include, but are not limited to, certain phenylenedialkanoate esters, CE-2072, UT-77, and triazole peptoids. Examples of analogues are TLCK (tosyl-L-lysine chloromethyl ketone) or TPCK (tosyl-L-phenylalanine chloromethyl ketone) or any combination thereof.

### BRIEF DESCRIPTION OF THE DRAWINGS

The following drawings form part of the present specification and are included to further demonstrate certain embodiments disclosed herein. Embodiments may be better understood by reference to one or more of these drawings in combination with the detailed description of specific embodiments presented herein.

FIG. 1 represents an exemplary comparison of AAT (Aralast™ or Zemaira™) and their respective effects on HIV production in infected peripheral blood mononuclear cells (PBMC).

FIGS. 2A and 2B represents an exemplary histogram of Heat-inactivated AAT (AAAT/HJ AAT) or native AAT on interleukin 8 (IL-8, 2A) or (IL-6, 2B) induction in human primary fibroblasts.

FIG. 3 represents an exemplary graph demonstrating HI AAT and AAT Elastase binding activity. Elastase alone is a control.

FIGS. 4A and 4B represents an exemplary histogram of the effects of AAT (4A left panel, solid bars) or HI AAT (4A right panel, open bars) at 0, 6, 4, 2 and 1 mg/ml on HIV production represented by p24 production (pg/ml) in stimulated U1 cells. FIG. 4B represents an exemplary histogram of the effects of AAT (5 mg/ml, 0.8 mg/ml) or HI AAT (striped bar, 5 mg/ml, 0.8 mg/ml) on HIV production represented by p24 production (pg/ml) in stimulated U1 cells.

FIG. 5 represents an exemplary histogram of AAT (open bars, left) and HI AAT (open bars, right) on lethal toxin-induced cytotoxicity (LDL OD units) in Raw 264.7 cells. A control is represented by a solid bar.

FIG. 6 represents an exemplary experiment of post-toxin treatment of mice with and without heat-inactivated AAT.

FIG. 7 represents an exemplary experiment of post-toxin treatment of mice with heat-inactivated AAT or placebo.

### DESCRIPTION OF ILLUSTRATIVE EMBODIMENTS

### Definitions

As used herein, “a” or “an” may mean one or more than one of an item. As used herein, “about” can mean plus or minus 10%, for example, about 10 minutes can mean from 9 to II minutes.

As used herein “analog of alpha-1-antitrypsin” may mean a compound having alpha-1-antitrypsin-ike activity other than serine protease inhibitor activity. In one embodiment, an analog of alpha-1-antitrypsin is a functional derivative of alpha-1-antitrypsin. In a more particular embodiment, an analog of alpha-1-antitrypsin is a compound with no significant serine protease inhibitor activity.

### DETAILED DESCRIPTION

In the following sections, various exemplary compositions and methods are described in order to detail various embodiments of the invention. It will be obvious to one skilled in the art that practicing the various embodiments does not require the employment of all or even some of the specific details outlined herein, but rather that concentrations, times and other specific details may be modified through routine experimentation. In some cases, well known methods, or components have not been included in the description.

Embodiments herein provide for methods and compositions for treating a subject having a medical disorder or exposed to a bacterial organism capable of causing a medical disorder. In accordance with these embodiments, the disorder may include, but is not limited to, a viral infection, a bacterial infection, or a combination thereof.

Certain embodiments concern compositions for treating a subject having a medical disorder. In accordance with these embodiments, the composition may include, alpha-1 antitrypsin, a fragment thereof, an analog thereof, or fusion molecule thereof, having no significant serine protease inhibition activity. In other embodiments, a composition may further include, but is not limited to, an anti-inflammatory agent, an immunosuppressive agent, an immunomodulatory agent, an anti-microbial agent, an anti-viral agent, an anti-bacterial agent, and a combination thereof.

Other embodiments herein concern methods of treating a subject with a medical disorder including administering to the subject in need of such a treatment a therapeutically effective amount of a composition including but not limited alpha-1 antitrypsin, a fragment thereof, an analog thereof, or fusion molecule thereof, having no significant serine protease inhibition activity. In accordance with these embodiments, the disorder can be a viral-infection related disorder infection, a bacterial-infection related disorder or a combination thereof. In other embodiments, the medical disorder can include, but is not limited to, sepsis, septic shock, Acute Respiratory Distress Syndrome (ARDS), reperfusion arrhythmias, ischemia/reperfusion heart injury, congestive heart failure, cardiac ischemia, stroke, cerebral vascular disorder, influenza, acute liver failure, chronic liver failure, and a common cold.

### Influenza

In certain embodiments, a medical disorder can include a viral infection for example, influenza such as influenza A or B. In accordance with these embodiments, a subject having been exposed to or having influenza can be administered a therapeutically effective amount of a composition contemplated herein. In one example, a composition can include, but not limited to, AAT, pre-treated in such a manner that no significant serine protease inhibitor activity remains. In one example, AAT is heated to reduce or eliminate serine protease inhibitor activity.

### Human Immunodeficiency Virus (HIV)

In certain embodiments, a medical disorder can include a viral infection for example, HIV or AIDS. In the course of the AIDS progression, many measurable clinical parameters including AAT progressively increase. In one study, it was demonstrated that AAT levels were affected in 40% of HIV-positive patients with cryptosporidial infections and none of 12 HIV-positive patients with non-cryptosporidial diarrhea. The incidence of abnormal AAT phenotypes was 16.3% in the homosexual group which was significantly different (p less than 0.03) than the 8.7% in the heterosexual group. There was no difference in the phenotype distribution between homosexuals who were anti-HIV antibody reactive and those who were non-reactive. Faecal AAT concentration was reflective of abnormal pancreatic function of paediatric HIV infection. Patients with HIV infection are known to acquire an obstructive pulmonary disease with clinical similarity to emphysema. AAT levels measured in these patients were in the lower normal range. Despite observing these clinical findings in 42% of consecutive HIV-infected patients in the clinic, no evidence of current or of previous opportunistic infection was detected. Bronchoalveolar lavage fluid obtained in a subset of these patients contained TNF and free radicals, indicating inflammation.

In certain embodiments, compositions including, but not limited to, AAT and derivatives thereof, having no significant serine protease activity, are useful for inhibition of HIV. In accordance with these embodiments, methods herein may concern treating a subject having HIV infection or having been exposed to HIV by administering to the subject in need of such a treatment a therapeutically effective amount of a composition including, but not limited to, AAT having no significant serine protease inhibitor activity. Another embodiment includes regulating virus release in a subject having an HIV infection by administering compounds having AAT activity other than serine protease inhibitor activity either alone or in combination with other anti-HIV compounds.

A treatment contemplated herein may include a treatment administered to a subject in need thereof multiple times daily, twice daily, daily, bi-weekly, weekly or other treatment regimen. In addition, a treatment for a subject having an HIV infection can also include any other treatment known in the art. Other treatments can include, but are not limited to, anti-viral compounds, anti-HIV compounds, reverse transcriptase inhibitor and a combination thereof.

In certain embodiments, methods of treatment contemplated herein can be used for reducing or preventing delivery of viral nucleic acid molecules into the nucleus of a mammalian host, as well as, methods for reducing or preventing the exit of a virion particle from a mammalian host harboring an agent of a viral infection. Thus, treatments contemplated herein may both reduce infection in a mammalian host but may also reduce or prevent spread of the infection. In certain particular embodiments, these processes are mediated by AAT activity other than AAT-associated serine protease inhibitor activity. These may be counteracted by administering a pharmacologically effective amount of a substance exhibiting mammalian α1-antitrypsin (AAT) having no significant serine protease inhibitor activity. In accordance with these methods, a post-exposure prophylaxis can be administered to a subject in need of such a treatment in order to block establishment of productive infection in a mammal exposed to FHV-contaminated fluids. Fluids contemplated to harbor HIV can include, for example, blood, saliva, semen, sweat, urine, vaginal secretion, tears, and other body fluids. In other embodiments, these methods and treatment compositions may be effective in reducing or preventing mother-to-child HIV transmission during pregnancy. In other embodiments, AAT having no significant serine protease inhibitor activity may be added to a syringe or other container to reduce or prevent transmission of HIV.

It is contemplated herein that assays for assessing the various activities of AAT or AAT-like molecules can be used. In one particular embodiment, AAT and similarly active compounds may be identified by a series of assays wherein a compound will exhibit inhibitory activity versus a control in an assay. In one exemplary method, an assay includes blocking interleukin-18 or IL-18-induced HIV production in for example, U1 monocytic cells. Other assays can include but are not limited to blocking stimulants such as IL-6, NaCl, LPS, TNF, and other HIV stimulants known in the art. In addition, other assays may involve a MAGI-CCR-5 cell assay and a PBMC assay as previously described.

Other viral infections contemplated herein include, but are not limited to, viral infections that are caused/facilitated at least in part by a deficiency in AAT levels or by a dysfunction of AAT. It is contemplated herein that a subject having a deficiency in AAT levels or a dysfunction of AAT can be treated by any composition contemplated herein.

Other agents contemplated of use in combination with AAT or an AAT-like molecule having no significant serine protease inhibitor activity are contemplated. In one embodiment, a method for treating HIV infection in a subject can include administering a therapeutically effective combination of (a) one or more compounds disclosed herein and (b) one or more compounds selected from the group consisting of HIV reverse transcriptase inhibitors and HIV protease inhibitors. Accordingly reverse transcriptase inhibitor can be selected from a group including nucleoside RT inhibitors: Retrovir (AZT/zidovudine; Glaxo Wellcome); Combivir (Glaxo Wellcome); Epivir (3TC, lamivudine; Glaxo Wellcome); Videx (ddI/didanosine; Bristol-Myers Squibb); Hivid (ddC/zalcitabine; Hoffmann-LaRoche); Zerit (d4T/stavudine; Bristol-Myers Squibb); Ziagen (abacavir, 1592U89; Glaxo Wellcome); Hydrea (Hydroxyurea[HO; nucleoside RT potentiator from Bristol-Myers Squibb) or Non-nucleoside reverse transcriptase inhibitors (NNRTIs): Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead). Protease inhibitors (PI's) are selected from Fortovase (saquinavir; Hoffmann-La Roche); Norvir (ritonavir; Abbott Laboratories); Crixivan (indinavir; Merck & Company); Viracept (nelfinavir; Agouron Pharmaceuticals); Angenerase (amprenavir/14IW94; Glaxo Wellcome), VX-478, KNI-272, CGP-61755, U-103017 or any combination thereof.

Also contemplated is a method of preventing acquired or congenital deficiency of functional endogenous AAT levels in a subject susceptible to a viral infection that is mediated by AAT activity other than serine protease inhibitor activity by treating the subject with a pharmaceutical composition in a pharmaceutically acceptable carrier. In accordance with these methods, an effective amount of a substance exhibiting mammalian AAT or AAT-like activity (e.g. a substance having no significant serine protease inhibitor activity) and a thrombolytic agent such as tissue plasminogen activator, urokinase, streptokinase, or combinations or complexes thereof can be administered to the subject. The pharmaceutical composition may be a peptide or a small molecule, which exhibits AAT or AAT-like activity.

### Cytomegliovirus (CMV)

Cytomegalovirus (CMV) has a surface molecule HCMV gB that participates in viral entry into cells. A genetically engineered AAT variant, α1-PDX, was designed to confer inhibitory activity against furin. Extracellular α1-PDX blocked the production of infectious CMV in vitro, and the CMV inhibition was associated with reduced proteolytic activation of HCMV gB. Antiviral effect of AAT and of the genetically-engineered variant α1-PDX suggest a role for AAT in control of Influenza A and CMV production in vivo. In certain embodiments, it is contemplated that disclosed compositions and methods can be used to treat a subject having or exposed to CMV or influenza using a therapeutically effective amount of AAT or an AAT analog having no significant serine protease inhibitor activity.

A genetic defect in humans which causes AAT deficiency exists in certain populations. AAT-deficient subjects possess the mutant Z-type variant of AAT which contains a single point-mutation at amino acid 342 (Glu-Lys). Structurally abnormal AAT accumulates within liver cells, which are the primary source of circulating AAT. An associated defect in secretion from the liver results in serum concentrations of <15% of normal. This mutation affects 70,000-100,000 persons in the United States.

The National Heart, Lung, and Blood Institute (NHLBI) has a registry of AAT-deficient patients. These AAT deficient persons include a mixed population who receive, or do not receive, intravenous AAT replacement therapy. AAT allocation is not randomized in this registry, and patients receive AAT for various reasons. A recent publication describes the results of a voluntary computer-based (internet) survey of a subset of approximately 300 AAT-deficient patients followed in the NHLBI registry [Lieberman, 2000 #122]. One hundred forty-three of the 300 members of the internet subset (48%) responded to a voluntary questionnaire which included queries about subjective lung infections. A lung infection was defined as increased cough and sputum production, usually associated with change in sputum color, and with or without fever, use of antibiotics or hospitalization.

Ninety-five patients with AAT deficiency replied who were receiving AAT replacement, and 46 AAT deficient individuals who were not taking AAT replacement replied (1 patient in each group possessed the mixed AAT phenotype SZ). The 95 patients receiving AAT replacement therapy were asked to compare the yearly incidence of lung infections before and after initiation of AAT therapy. Compared to the yearly number of lung infections reported prior to initiation of AAT therapy, a significant reduction in the number of lung infections was reported following the initiation of AAT therapy. Many patients also believed that head colds and flu were less frequent following the initiation of AAT replacement. In a separate comparison, the 95 members of the NHLBI cohort who received AAT replacement therapy were compared with the 46 who did not receive replacement therapy. The group receiving AAT therapy reported fewer yearly lung infections than did the group not receiving therapy.

Characteristics of the AAT-treated and non-treated groups were assessed for comparability in age, sex and smoking status. Taken together, the above results in vitro and in the NHLBI AAT-deficient registry subset suggest the possibility that AAT is a natural inhibitor of Influenza A virus and CMV. Furthermore, investigation of AAT-deficient populations may provide a useful means of studying the association between AAT and infection with these viruses in vivo.

In one particular study, human subjects were assessed who have undergone lung transplantation. Since AAT-deficient patients often acquire severe emphysema which can require lung transplantation, these patients were studied epidemiologically. Following transplantation, the members of this study followed a strict protocol of medical management, and each receives frequent medical assessment. An extensive and detailed database is maintained on each of these patients. The database was inspected to specifically evaluate the relationship between AAT deficiency and infection with Influenza A virus or with CMV. AAT deficient patients were found to have substantial and statistically significant increases in infection with influenza A (Flu) and with cytomegalovirus (CMV). These data establish AAT deficiency as a novel risk factor for infections with Flu and with CMV. It is contemplated herein that any composition may be administered to a subject having such a deficiency identified above.

### Mycobacteria

In other embodiments, medical disorders contemplated herein may include mycobacterial-associated diseases or disorders. In accordance with these embodiments, methods for treating a subject having or suspected of getting a mycobacterial infection are contemplated. In certain embodiments, a mycobacterial infection contemplated herein can include, but are not limited to, those mycobacterial diseases or disorders caused by mycobacteria from the genus mycobacterium that includes Mycobacterium tuberculosis (M. tuberculosis), M. bovis, M. leprae, M. avium-intracellulare, M. chelonei (also known as borstelense and abscessus), M. africanum, M. marinium (also known as balnei and platypoecilus), M. buruli (also known as ulcerans), M. fortuitum (also known as giae, minetti, and ranae), M. haemophilum, M. intracellulare, M. kansasii (also known as luciflavum), M. littorale (also known as xenopi), M. malmoense, M. marianum (also known as scrofulaceum and paraffinicum), M. simiae, M. szulgai, M. ulcerans, M. avium (also known as brunense), M. flavascens, M. lepraemurium, M. microti, and M. paratuberculosis (which is the causative agent for Johne's Disease, and a possible cause of Crohn's disease), M. gordonae (also known as aquae), M. gastri, M. phlei (also known as moelleri and as timothy bacillus), M. nonchromogenicum, M. smegmatis, M. terrae, M. triviale, and M. vaccae.

In another embodiment, a mycobacterium infection of macrophages may be reduced or inhibited. These mycobacterium can be from the genus that includes non-tuberculosis mycobacteria that are divided into four groups comprising Runyon groups, selected from the group consisting of Group I (slow-growing photochromogens), Group II (slow-growing scotochromogens), Group III (slow-growing nonphotochromogens), and Group IV (rapidly-growing mycobacteria).

In certain embodiments, compositions including, but not limited to, AAT and derivatives thereof, having no significant serine protease inhibitor activity, are useful for inhibition of mycobacterium or mycobacterium infection. In accordance with these embodiments, methods herein can concern treating a subject having mycobacterium or mycobacterium infection or having been exposed to mycobacteria by administering to the subject in need of such a treatment a therapeutically effective amount of a composition including, but not limited to, AAT having no significant serine protease inhibitor activity. One particular embodiment concerns regulating mycobacterial infection by administering compounds having AAT activity other than serine protease inhibitor activity either alone or in combination with other compounds, for example, other anti-bacterial compounds.

### Bacillus Anthracis and Anthrax Toxin

Anthrax toxin, produced by the gram positive rod-shaped aerobic, spore-forming bacterium Bacillus anthracis, is the toxic virulence factor secreted by this organism. B. anthracis is often considered for use as a biological weapon due to the potency of the secreted exotoxin, and to the capacity of the bacterium to form dormant spores which resist harsh environmental conditions. Sporulation enables ready transport and distribution of large quantities of toxin-producing bacteria. The toxin is actually a composite consisting of 3 separate secreted proteins from the bacterium. The three proteins are protective antigen (PA), lethal factor (LF), and edema factor (EF). LF and EF directly damage cells and are thought to cause disease due to anthrax toxin exposure. PA is crucial to the virulence of anthrax toxin, since the PA molecule is designed to import both LF and EF inside the membranes of cells. In the absence of PA-induced intracellular transport, anthrax toxin is unable to effect tissue destruction, since LF and EF only function from within the cell.

### Clinical Symptoms of Anthrax

Anthrax occurs as three general clinical entities: i) inhalation, ii) cutaneous, and iii) gastrointestinal forms.

1.) Inhalation anthrax is the deadliest form of the disease, and it is the one most likely to be involved in a bioweapons altercation or accident. Usually, an infected person inhales anthrax spores serendipitously, or during a bioweapons attack. Following a 1-6 day incubation period, a biphasic illness ensues. Initially, non-specific malaise/fever/dry cough/myalgias, and chest pains occurs. The second phase, 2-3 days after the first phase, consists of progression of the constitutional non-specific findings listed above, an addition to ventilatory compromise, sweating, widening of the mediastimum on radiographic studies, and edema of the neck and chest. This stage of illness is characterized by a necrotizing mediastinal lymphadenitis. This second stage of disease can rapidly progress to shock and death within 2 days, and mortality rates of up to 80% have been reported. The mechanism of death in animal models appears to be enhanced production of pro-inflammatory cytokines, especially IL-1. In one embodiment, disorders associated with inhalation anthrax may be reduced or prevented by administering to a subject in need of such a treatment, a composition including AAT, a fragment thereof, an analog thereof, or a compound with AAT-like activity having no significant serine protease inhibitor activity by for example, inhalation.

2) Cutaneous anthrax is the most common form of anthrax infection in humans. For example, after exposure to anthrax spores, regions of skin eruptions (cuts, abrasions, etc.), present an environment that allows anthrax organisms to emerge from the spore state, to grow, replicate, and produce anthrax toxin. Within 1 week, an area of anthrax innoculation develops a painless papule. Vesicles then form on or near the papule over the ensuing 1-2 days, followed shortly by development of fever and malaise, and a non-pitting edema surrounding the skin lesion that is due to toxin activity. The original lesion ruptures to form necrotic ulceration and enlargement resulting in formation of the eschar that characterizes cutaneous anthrax infection. In the absence of therapy, this disease carries a 20% morality. For those who recover, the eschar sloughs off in 1-2 weeks. In certain embodiments, a method for treating a subject having or exposed to cutaneous anthrax is contemplated herein. In accordance with these embodiments, a topical/cream preparation having AAT or a compound with AAT-like activity having no significant serine protease inhibitor activity can be used. Parenteral compositions, including but not limited to, AAT or a compound with AAT-like activity, but having no significant serine protease inhibitor activity, can also be co-administered in the event that systemic symptoms emerge, or such parenteral therapy can be administered prophylactically for anthrax that appears clinically to be localized to the skin.

3) Gastrointestinal anthrax appears after ingestion of anthrax spores. After 2-5 days, one develops nausea/vomiting/fever, and abdominal pain. Bloody diarrhea rapidly ensues, and an “acute abdomen” manifests. The pathology within the abdomen includes mucosal ulcerations. Also, hemorrhagic mesenteric lymphadenitis develops, and this is again consistent with selective activation of the anthrax toxin in serine protease-inhibitor deficient microenvironments. This disease carries a mortality rate of 50%. Certain embodiments contemplated herein concern administering a compositions to a subject having or been exposed to gastrointestinal anthrax. In accordance with these embodiments, the composition can include, but is not limited to, AAT or a compound with AAT-like activity, but having no significant serine protease inhibitor activity.

### Pneumonia

In certain embodiments, a therapeutically effective amount of AAT having no significant serine protease inhibitor activity can be administered to a subject having or exposed to bacterial, viral, fungal, or parasitic pneumonia.

In certain embodiments, the reduction, prevention or inhibition of infection or side effects thereof associated with one or more of each of the above-recited conditions may be about 10-20%, 30-40%, 50-60%, or more reduction or inhibition due to administration of the disclosed compositions.

In certain particular embodiments, compositions and methods herein can concern treating a subject having a fungal infection. In accordance with these methods the subject can be administered a therapeutically effective amount including, but not limited to, a composition of AAT or a composition with AAT-like activity, but having no significant serine protease inhibitor activity.

### Proteins and Polypeptides

One embodiment pertains to isolated proteins, and biologically active portions thereof, as well as polypeptide fragments. In one embodiment, the native polypeptide can be isolated from cells or tissue sources by an appropriate purification scheme using standard protein purification techniques. In certain embodiments, the native polypeptide may be heated or otherwise treated to reduce or eliminate serine protease inhibitor activity. In certain particular embodiments, serine protease inhibitor activity is reduced where no significant activity remains. In another embodiment, polypeptides contemplated herein are produced by recombinant DNA techniques. Alternative to recombinant expression, a polypeptide can be synthesized chemically using standard peptide synthesis techniques. Any of the peptide or protein molecules contemplated of use in compositions disclosed herein can be compositions having no significant serine protease inhibitor activity. For example, AAT compositions may be treated in order to reduce or eliminate serine protease inhibitor activity or an AAT polypeptide may be isolated wherein the polypeptide has reduced or no significant serine protease inhibitor activity.

Recombinant unmodified and mutant variants of AAT produced by genetic engineering methods are also known (U.S. Pat. No. 4,711,848). The nucleotide sequence of human AAT and other human AAT variants have been disclosed. In certain embodiments, these nucleotide sequence or amino acid sequences may be used as starting material to generate all of the AAT amino acid variants and amino acid fragments depicted herein, using recombinant DNA techniques and methods known to those of skill in the art. It is contemplated that any of these mutants or variants have no significant serine protease inhibitor activity.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium. When the protein is produced by chemical synthesis, it is preferably substantially free of chemical precursors or other chemicals. For example, such preparations of the protein have less than about 30%, 20%, 10%, 5% (by dry weight) of chemical precursors or compounds other than the polypeptide of interest.

Biologically active portions of a polypeptide can include polypeptides including amino acid sequences sufficiently identical to or derived from the amino acid sequence of the protein (e.g., the amino acid sequence, which exhibit at least one activity of the corresponding full-length protein except serine protease inhibition activity). A biologically active portion of a protein can be a polypeptide, which is, for example, 5, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

In certain embodiments, polypeptides can include a polypeptide having an amino acid sequence corresponding to the carboxy terminus of AAT or AAT allele. Other useful proteins are substantially identical (e.g., at least about 45%, preferably 55%, 65%, 75%, 85%, 95%, or 99%) to any portion of the carboxy terminus, and retain the functional activity of the protein of the corresponding naturally-occurring protein other than serine protease inhibitor activity yet differ in amino acid sequence due to natural allelic variation or mutagenesis.

Compounds herein can be used as therapeutic agents in the treatment of a physiological (especially pathological) condition caused in whole or part, by excessive serine protease activity. In addition, a physiological (especially pathological) condition can be inhibited in whole or part. Peptides contemplated herein may be administered as free peptides or pharmaceutically acceptable salts thereof. Peptides may be administered to a subject as a pharmaceutical composition, which, in most cases, will include the peptide and/or pharmaceutical salts thereof with a pharmaceutically acceptable carrier.

When utilizing BLAST, Gapped BLAST, and PSI-Blast programs, the default parameters of the respective programs (e.g., XBLAST and NBLAST) can be used. See for example, http://www.ncbi.nlm.nih.gov.

Varients of the polypeptides are contemplated herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein may inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein contemplated herein which function as either agonists (mimetics) or as antagonists can be identified by screening combinatorial libraries of mutants, e.g., truncation mutants, of the protein of the invention for agonist or antagonist activity.

### Fusion Polypeptides

In other embodiments, compounds having AAT activity other than serine protease inhibitor activity, such as AAT and/or analog thereof, may be part of a fusion polypeptide. In one example, a fusion polypeptide may include AAT (e.g. mammalian α1-antitrypsin) or an analog thereof and a different amino acid sequence that may be heterologous to AAT or analog substance, having no significant serine protease inhibitor activity.

In yet other embodiments, a fusion polypeptide (e.g., IgG or fragment thereof) contemplated of use in methods herein can additionally include an amino acid sequence that is useful for identifying, tracking or purifying the fusion polypeptide, e.g., a FLAG or HIS tag sequence. The fusion polypeptide can include a proteolytic cleavage site that can remove the heterologous amino acid sequence from the compound capable of serine protease inhibition, such as mammalian AAT or analog thereof.

In one embodiment, fusion polypeptides can be produced by recombinant DNA techniques. Alternative to recombinant expression, a fusion polypeptide of the invention can be synthesized chemically using standard peptide synthesis techniques. In addition, a fusion polypeptide disclosed herein can include a pharmaceutically acceptable carrier, excipient or diluent.

In certain embodiments, a fusion protein can include a heterologous sequence derived from a member of the immunoglobulin protein family, for example, an immunoglobulin constant region, e.g., a human immunoglobulin constant region such as a human IgG1 constant region. A fusion protein can, for example, include a portion of AAT, analog thereof fused with the amino-terminus or the carboxyl-terminus of an immunoglobulin constant region, by methods known in the art. In accordance with these embodiments, the FcR region of the immunoglobulin may be either wild-type or mutated. In certain embodiments, it may be desirable to utilize an immunoglobulin fusion protein that does not interact with an Fc receptor and does not initiate ADCC reactions. In such instances, the immunoglobulin heterologous sequence of the fusion protein can be mutated to inhibit such reactions. See for example, U.S. Pat. No. 5,985,279 and WO 98/06248.

In yet another embodiment, AAT, analog thereof, polypeptide fusion protein can be a GST fusion protein in which is fused to the C-terminus of GST sequences. Fusion expression vectors and purification and detection means are known in the art.

Expression vectors can routinely be designed for expression of a fusion polypeptide of the invention in prokaryotic (e.g., E. coli) or eukaryotic cells (e.g., insect cells (using baculovirus expression vectors), yeast cells or mammalian cells) by means known in the art.

Expression of proteins in prokaryotes may be carried out by means known in the art. Such fusion vectors typically serve three purposes: 1) to increase expression of recombinant protein; 2) to increase the solubility of the recombinant protein; and 3) to aid in the purification of the recombinant protein by acting as a ligand in affinity purification.

In yet another embodiment, a nucleic acid of the invention is expressed in mammalian cells using a mammalian expression vector as described in the art. In another embodiment, a recombinant mammalian expression vector is capable of directing expression of the nucleic acid in a particular cell type (e.g., tissue-specific regulatory elements are used to express the nucleic acid) such as pancreas-specific promoters and mammary gland-specific promoters. A host cell can be any prokaryotic (e.g., E. coli) or eukaryotic cell (e.g., insect cells, yeast or mammalian cells). Vector DNA can be introduced into prokaryotic or eukaryotic cells via conventional transformation or transfection techniques.

Some embodiments contemplated herein include adding and/or deleting groups from alpha-1 antitrypsin, a fragment thereof, an analog thereof, or fusion molecule thereof. In accordance with these embodiments, these molecules may be deglycoslyated prior to use in methods disclosed herein. In other embodiments stabilizing compounds may be linked to the molecules to increase stability when used in methods disclosed herein. For example, PEG (polyethylene glycol) may be added to increase stabilization of compositions contemplated herein.

### Other Agents

Any of the embodiments detailed herein may further include one or more a therapeutically effective amount of anti-microbial drugs, anti-inflammatory agent, immunomodulatory agent, or immunosuppressive agent or combination thereof.

Examples of anti-bacterial agents include, but are not limited to, penicillins, quinolonses, aminoglycosides, vancomycin, monobactams, cephalosporins, carbacephems, cephamycins, carbapenems, and monobactams and their various salts, acids, bases, and other derivatives.

Anti-fungal agents contemplated of use herein can include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents contemplated of use herein can include, but are not limited to, valgancyclovir, amantadine hydrochloride, rimantadin, acyclovir, famciclovir, foscamet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents contemplated of use herein can include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole, (sulfamethoxazole/trimethoprim), and pentamidine isethionate.

Immunomodulatory agents can include for example, agents which act on the immune system, directly or indirectly, by stimulating or suppressing a cellular activity of a cell in the immune system, (e.g., T-cells, B-cells, macrophages, or antigen presenting cells (APC)), or by acting upon components outside the immune system which, in turn, stimulate, suppress, or modulate the immune system (e.g., hormones, receptor agonists or antagonists, and neurotransmitters); other immunomodulatory agents can include immunosuppressants or immunostimulants. Anti-inflammatory agents can include, for example, agents which treat inflammatory responses, tissue reaction to injury, agents which treat the immune, vascular, or lymphatic systems or any combination thereof.

Anti-inflammatory or immunomodulatory drugs or agents contemplated of use herein can include, but are not limited to, interferon derivatives, e.g., betaseron, β-interferon; prostane derivatives, iloprost, cicaprost; glucocorticoids such as cortisol, prednisolone, methylprednisolone, dexamethasone; immunosuppressive agents such as cyclosporine A, FK-506, methoxsalene, thalidomide, sulfasalazine, azathioprine, methotrexate; lipoxygenase inhibitors, e.g., zileutone, MK-886, VY-50295, SC-45662, SC-41661A, BI-L-357; leukotriene antagonists; peptide derivatives for example ACTH and analogs; soluble TNF (tumor necrosis factor)-receptors; TNF-antibodies; soluble receptors of interleukines, other cytokines, T-cell-proteins; antibodies against receptors of interleukins, other cytokines, and T-cell-proteins.

Other agents of use in combination with compositions herein can be molecules having serine protease inhibitor activity. For example serine protease inhibitors contemplated of use herein can include, but are not limited to, leukocyte elastase, thrombin, cathepsin G, chymotrypsin, plasminogen activators, and plasmin.

In certain embodiments, a composition may include one or more peptides of an AAT or AAT analog where the peptide(s) have similar activity to an AAT or AAT analog having no significant serine protease inhibitor activity. In each of the recited methods, an α1-antitrypsin (e.g. mammalian derived) substance having no significant serine protease inhibitor activity contemplated for use within methods disclosed herein can include a series of peptides including carboxyterminal or amino terminal amino acid peptides corresponding to or derived from any AAT molecule contemplated herein. In certain embodiments, the peptides can be 5 or 10 or 20 or 30 or 40 or more amino acids in length.

In other particular embodiments herein, AAT peptides contemplated for use in the compositions and methods of the present invention are also intended to include any and all of those specific AAT peptides depicted supra. Any combination of consecutive amino acids simulating AAT having no significant serine protease inhibitor activity may be used, such as amino acids 2-12, amino acids 3-14, 4-16, 5-20, 10-30, etc.

In addition, other combination compositions of methods disclosed herein can include certain antibody-based therapies. Non-limiting examples include, polyclonal anti-lymphocyte antibodies, monoclonal antibodies directed at the T-cell antigen receptor complex (OKT3, TIOB9), monoclonal antibodies directed at additional cell surface antigens, including interleukin-2 receptor alpha. In certain embodiments, antibody-based therapies may be used as induction therapy in combination with the compositions and methods disclosed herein.

Subjects contemplated herein can include human subjects, or other subjects such as non-human subjects, including but not limited to, primates, dogs, cats, horses, cows, pigs, guinea pigs, birds and rodents.

### Pharmaceutical Compositions

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, antibody, or anti-viral agent) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, the compound (e.g. pharmaceutical chemical, protein, gene, antibody, or anti-viral agent) may be administered to a subject in need thereof subcutaneously, intravenously, by oral administration, inhalation, transdermally, intravaginally, topically, intranasally, rectally or a combination thereof. Depending on the route of administration, the active compound may be coated in a material to protect the compound from the degradation by enzymes, acids and other natural conditions that may inactivate the compound. In a preferred embodiment, the compound may be orally administered. In another preferred embodiment, the compound may be administered intravenously. In one particular embodiment, the compound may be administered intranasally, such as inhalation.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used. In all cases, the composition can be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g. a compound that has reduced or no significant serine protease inhibitor activity) in the required amount in an appropriate solvent with one or a combination of ingredients enumerated above, as required, followed by filter sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art.

Solutions of the active compounds as free-base or pharmacologically acceptable salts can be prepared and suitably mixed with for example, a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Prolonged absorption of the injectable or ingestible compositions can be brought about by compositions of agents delaying absorption, for example, aluminum monostearate, gelatin or the like. In other embodiments, a composition contemplated herein can be in the form of a slow or time-released particle or capsule such as microparticles, for example, microbeads or a microgel. In accordance with these embodiments, a microparticle can contain a composition disclosed herein and once the microparticles are introduced to a subject in need thereof the composition can be released upon targeting a specific region, in timed intervals or as the microparticles degrade. These methods are known in the art and are contemplated herein.

Therapeutic agents may be formulated within a mixture to include about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. In certain embodiments, oral pharmaceutical compositions can include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

Pharmaceutical compositions are administered in an amount, and with a frequency, that is effective to inhibit or alleviate side effects of a transplant and/or to reduce or prevent rejection. The precise dosage and duration of treatment may be determined empirically using known testing protocols or by testing the compositions in model systems known in the art and extrapolating therefrom. Dosages may also vary with the severity of the condition. A pharmaceutical composition is generally formulated and administered to exert a therapeutically useful effect while minimizing undesirable side effects. In general, an oral dose ranges from about 200 mg to about 1000 mg, which may be administered for example, 1 to 3 times per day.

It is contemplated that, for a particular subject, specific dosage regimens may be adjusted over time according to need. A preferred dose for administration can be anywhere in a range between about 0.01 mg and about 100 mg per ml of biologic fluid of treated subject. In one particular embodiment, the range can be between 1 and 100 mg/kg which can be administered daily, every other day, biweekly, weekly, monthly etc. In another particular embodiment, the range can be between 10 and 75 mg/kg introduced weekly to a subject. A therapeutically effective amount of AAT, peptides, or drugs that have similar activities as AAT or peptides other than serine protease inhibitor activity can be also measured in molar concentrations and can range between about 1 nM to about 2 mM.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent.

Liposomes can be used as a therapeutic delivery system and can be prepared in accordance with known laboratory techniques. In addition, dried lipids or lyophilized liposomes prepared as previously described may be reconstituted in a solution of active agent (e.g. nucleic acid, peptide, protein or chemical agent), and the solution diluted to an appropriate concentration with a suitable solvent known to those skilled in the art. The amount of active agent encapsulated can be determined in accordance with standard methods.

In a one embodiment, a nucleic acid (e.g. AAT or analogs thereof) and the lipid dioleoylphosphatidylcholine may be employed. For example, nuclease-resistant oligonucleotides may be mixed with lipids in the presence of excess t-butanol to generate liposomal-oligonucleotides for administration.

Pharmaceutical compositions containing AAT, analog thereof, or a functional derivative thereof may be administered to individuals, particularly humans, for example by subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration is accomplished by application of a cream, rinse, gel, etc. capable of allowing the inhibitors of serine proteases to penetrate the skin and enter the blood stream. In addition, osmotic pumps may be used for administration. The necessary dosage will vary with the particular condition being treated, method of administration and rate of clearance of the molecule from the body.

In each of the aforementioned compositions and methods, a compound having no significant serine protease inhibitor activity but having other α1-antitrypsin activity or analog thereof may be used in a single therapeutic dose, acute manner or a chronic manner to treat episodes or prolonged bouts, respectively, in reducing or eliminating a medical disorder contemplated herein.

### AAT

Human AAT is a single polypeptide chain with no internal disulfide bonds and only a single cysteine residue normally intermolecularly disulfide-linked to either cysteine or glutathione. One reactive site of AAT contains a methionine residue, which is labile to oxidation upon exposure to tobacco smoke or other oxidizing pollutants. Such oxidation reduces the elastase-inhibiting activity of AAT; therefore substitution of another amino acid at that position, e.g., alanine, valine, glycine, phenylalanine, arginine or lysine, produces a form of AAT which is more stable. AAT can be represented by the following formula:

Extra hepatic sites of AAT production include neutrophils, monocytes and macrophages, and the expression of AAT is inducible in response to LPS, TNFα, IL-1 and IL-6 in various cell types. Deficiency in AAT is associated with immune dysfunctional conditions such as rheumatoid arthritis and systemic lupus erythematosus.

Serine protease inhibitor molecules, which may be used in combination with compositions disclosed herein may include compounds disclosed in the following: WO 98/20034 disclosing serine protease inhibitors from fleas; WO98/23565 disclosing aminoguanidine and alkoxyguanidine compounds useful for inhibiting serine proteases; WO98/50342 disclosing bis-aminomethylcarbonyl compounds useful for treating cysteine and serine protease disorders; WO98/50420 cyclic and other amino acid derivatives useful for thrombin-related diseases; WO 97/21690 disclosing D-amino acid containing derivatives; WO 97/10231 disclosing ketomethylene group-containing inhibitors of serine and cysteine proteases; WO 97/03679 disclosing phosphorous containing inhibitors of serine and cysteine proteases; WO 98/21186 benzothiazo and related heterocyclic inhibitors of serine proteases; WO 98/22619 disclosing a combination of inhibitors binding to P site of serine proteases with chelating site of divalent cations; WO 98/22098 disclosing a composition which inhibits conversion of pro-enzyme CPP32 subfamily including caspase 3 (CPP32/Yama/Apopain); WO 97/48706 disclosing pyrrolo-pyrazine-diones; and WO 97/33996 disclosing human placental bikunin (recombinant) as serine protease inhibitor.

### Kits

Other embodiments concern kits for use with compositions and methods described above. In certain embodiments, small molecules, proteins or peptides may be employed for use in any of the disclosed methods. In addition, other agents such as anti-bacterial agents, immunosuppressive agents, anti-inflammatory agents, and/or anti-viral agents may be provided in the kit. The kits can include, a suitable container (e.g. vial, syringe, bottle, tube,) a protein or a peptide or analog agent, and optionally one or more additional agents.

The kits may further include a suitably aliquoted composition of the encoded protein or polypeptide antigen, whether labeled or unlabeled, as may be used to prepare a standard curve for a detection assay. In certain embodiments, a kit can include a composition including, but not limited to, AAT, AAT fragment, or an AAT analog or polypeptide, having no significant serine protease inhibitor activity.

A container of kits contemplated herein will generally include at least one vial, test tube, flask, bottle, syringe or other container means, into which an agent or agents may be placed, and preferably, suitably aliquoted. In accordance with these embodiments, a kit can contain AAT or an analog thereof having no significant serine protease inhibitor activity. Such containers may include injection or blow-molded plastic containers into which the desired vials are retained.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes may be made in the specific embodiments which are disclosed and still obtain a like or similar result without departing from the spirit and scope of the invention.

### General Procedure and Materials

In one exemplary method, AAT used in these studies is purified from the blood of healthy volunteers. AAT is purified to single-band homogeneity. The AAT protein is diafiltered into a diluent consisting of NaCl, sodium phosphate, pH 7.05. The AAT preparations are maintained at stock concentrations of 14-50 mg/ml and stored at −70.degree. C. until added to cultures. As a control AAT preparation that is different from the composition of the invention a commercially available Prolastin (Bayer's AAT) is used. Recombinant human interleukin (IL)-18 is obtained from Vertex Pharmaceuticals Inc., (Cambridge, Mass.). IL-6 and tumor necrosis factor (TNF) are obtained from R & D Systems, Minneapolis, Minn., endotoxin-free NaCl, and endotoxin (lipopolysaccharide, LPS) is obtained from Sigma (St. Louis, Mo.). Here is the heat inactivation graph.

### U1 Cells

Medium for monocytic U1 cell and MAGI-CCR5 cell cultures consists of RPMI 1640 medium purchased from Mediatech (Hermdon, Va.) containing 2.5 mM L-glutamine, 25 mM Hepes, 100 units/ml penicillin and streptomycin (GIBCO/BRL, Rockville, Md.) with 10% or 7.5% (vol/vol) heat-inactivated fetal bovine serum (FBS, GIBCO) for U1 cell and MAGI-CCR5 cell cultures, respectively. PBMC are cultured in R3 medium consisting of RPMI 1640 medium (Mediatech), 20% FBS (GIBCO), 100 units/ml penicillin and streptomycin (GIBCO) and 5% (vol/vol) IL-2 (Hemagen, Waltham, Mass.).

U1 monocytic cell assay. U1 cells can be obtained from the AIDS Research and Reference Reagent Program, National Institute of Allergy and Infectious Diseases, NIH. U1 cells are maintained in T-175 polystyrene flasks (Falcon, Becton Dickinson, Franklin Lakes, N.J.) in medium and used when in log phase growth. Cells are counted in a hemacytometer, examined for viability by Trypan blue exclusion (>95% for all experiments) and resuspended in fresh medium at 2×10⁶ per ml. Two-hundred fifty ml of cell suspension are added to wells of 24-well polystyrene tissue culture plates (Falcon), followed by the addition of medium or AAT to produce the final concentration to be tested in a volume of 450 ml. After 1.0 hr of incubation (37° C., 5% CO₂), 50 ml of medium (control) or stimulus diluted in medium are added to wells to produce the final concentration of stimulus to be tested. The final culture volumes are 500 ml and contained 1×10⁶ cells per ml. After 48 hr of incubation (37° C., 5% CO₂) 50 ml of 10% (vol/vol) Triton-X-100 is added to each culture (final concentration of 1% vol/vol), and cultures are frozen and thawed once. This is followed by assay for HIV p24 antigen by ELISA with a lower limit of detection of 31 pg/ml (NCI-Frederick Cancer Research and Development Center, Frederick, Md.). The disruption of cells due to the addition of Triton-X-100 and the freeze-thaw cycle produced cell lysates and enabled assessment of total (secreted and cell-associated) production of p24 antigen.

### Example 1

Blood Draw: In certain exemplary methods, first blood was drawn into syringes containing heparin (10 20 U/mL, or use commercial heparinized sterile tubes) and second, cells were separated. In one particular example, 1.0 mL blood provides 1×10⁶ PBMC and about 2.5×10⁶ PBMC per tube were used for these experimental examples.

Cell Separation can include for example:

a) 20 mL sterile saline is added to 50 ml polypropylene tubes.
b) Put 10 mL whole blood into each 50 mL polypropylene tube.
c) Underlay each tube with 10 mL ficoll hypaque using a pipette or a spinal needle, proceed at a rate of about 1 minute per underlay.
d) Centrifuge the tubes at 1,250 rpm (=400 g)×40 minutes at room temperature.
e) Harvest PBMC layers from 2 tubes using a 10 ml pipette and place into a fresh 50 ml polypropylene tube.
f) Fill tubes to 50 mL with saline.
g) Centrifuge tubes at 1,000 rpm×10 minutes at room temperature.
h) Decant supernatant.
i) Resuspend cells in 10 mL saline and combine all tubes into as few tubes as possible.
j) Fill tube(s) to 50 mL with saline.
k) Centrifuge tube(s) at 1,000 rpm×10 minutes at room temperature.
l) Decant supernatant.
m) Resuspend the cells with a pipette in EXACTLY 10 mL of saline.
n) Count cells in a hemacytometer (total #).
o) Add an additional 40 mL of saline to the tube(s); each now contains 50 mL liquid.
p) Centrifuge the tubes at 1,000 rpm×10 minutes at room temperature.
q) Decant supernatant.
r) Resuspend cells at 1×106/mL in sterile R3 tissue culture medium (RPMI 1640 medium with 20% [vol/vol] heat-inactivated fetal bovine serum, 5% [vol/vol] Interleukin (IL)-2 and penicillin 100 units/ml+streptomycin 100 μg/ml) supplemented 3.3 μg/ml PHA.

Third, cells were induced into blast phase by culture by incubation for 2 days (37° C., 5% CO2) in sterile tissue culture flasks.

Fourth, PBMC were then infected with HIV: After the 2 days of blasting/incubation, the cells were counted and the number of PBMC was determined for infecting with HIV. A cell suspension was aliquoted into a polypropylene tube, then centrifuged into a pellet. Then, the tubes are inverted right away, preserving the cell pellet: approximately 300 μl of liquid remains with the cell pellet. The virus of choice was added. For the X4/T tropic A018A strain, the PBMC was infected with 200 TCID50 per 1 million PBMC. For the R5/M tropic virus strain, 300 TCID50 per 1 million PBMC was used for infection. After adding the virus, the virus was resuspended vigorously with a pipetter and vortex as well. Then the cells were incubated in the 50 ml polypropylene tube (loose cap) for 3 hrs in an incubator. c) After 3 hrs of incubation, the infected PBMC were washed with RPMI or with PBS (resuspend with a vacuum pipetter), then centrifuge. No significant amount of virus remains after this step. d) The infected PBMC was resuspended at 2×106 per ml in non-blasting R3 medium=R3 medium as above but without PHA. (=RPMI+10% FCS+5% IL 2).

Fifth, the cell suspension was aliquoted into 24-well polystyrene plates at a final concentration of 1×10⁶ per ml. Sixth, a time zero sample was created by taking a 250 μl aliquot of cell suspension at 2×10⁶ cells per ml and add this into a 1.5 ml Eppendorf tube. Add to this 250 μl of medium and 50 μl of (10% vol/vol) Triton X 100. The sample is froze immediately at −70° C. and assay later for p24 antigen as the time 0 specimen. Seventh, 250 μl of cell suspension was added to each well with an additional 250 μl of R3 medium alone (Spontaneous, or AAT=0), or R3 that contains AAT (either Aralast® or Zemaira®) at twice the final desired concentrations. The final volume of each culture is 500 μl. Eighth, the tissue culture plates were incubated with cell cultures in an incubator (37° C., 5% CO2), for 4 days, then add 50 μl of 10% (vol/vol) Triton X 100 to make a final Triton X 100 concentration of 1% vol/vol. Finally HIV p24 antigen was quantified using an ELISA assay.

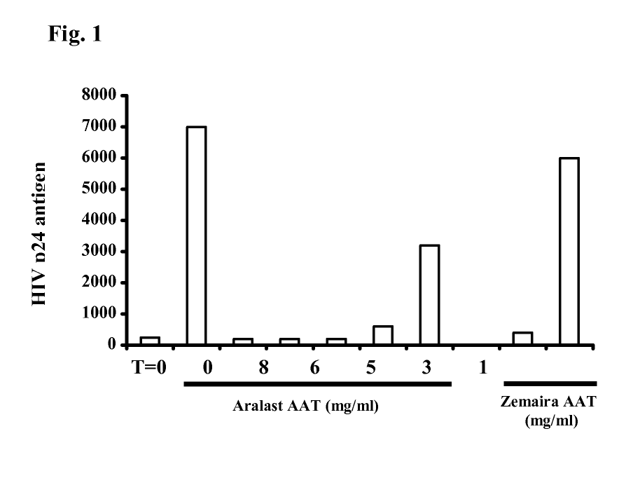

As demonstrated in exemplary FIG. 1, Aralast substantially induced HIV inhibition at all concentrations tested (compared to AAT=0 cultures), with nearly 100% suppression observed using Aralast at 3.0 8.0 mg/ml, and about 50% HIV suppression using Aralast at 1.0 mg/ml. In contrast, Zemaira AAT demonstrated minimal HIV suppression at 7.0 mg/ml, and near complete suppression was obtained at 15.0 mg/ml. In this exemplary method, there was a large difference in dose response demonstrating that Aralast is more potent than Zemaira as an inhibitor of HIV infection in primary PBMC. Since Aralast and Zemaira are quantified by biological activity (1.0 mg Aralast=1.0 mg Zemaira=1.0 mg of serine protease inhibitor activity), this experiment indicates that the ability of AAT to suppress HIV is independent of serine protease inhibition. If the serine protease inhibitor function of AAT accounts for the HIV suppression, Aralast and Zemaira would inhibit HIV production equivalently.

### Exemplary Procedures for Heat Inactivation (HI) of AAT

In another exemplary method, a predetermined volume (e.g. 2 mls) of a stock solution such as 20 mg/ml of AAT (e.g. Aralast) was placed in a test tube. The stock sample was heat treated in boiling water (95° C.) for 30 min. The solution was allowed to cool. Then the heated solution was transferred back to eppendorf tube(s). If any volume has boiled off (usually about 10%), the volume is replaced with a solution to near original volume using for example, PBS. Then the solution is tested for remaining serine protease activity using a serine protease inhibitor assay. It was demonstrated that no significant serine protease inhibitor activity could be detected for up to 3 days later (data not shown).

### Example 2

Elastase assay: In one example, an enzymatic assay of elastase biological activity (Bieth J, et. al 1974) was used to compare AAT and heat-inactivated (HI) AAT.

Elastase-induced hydrolysis of the N-Succinyl-Ala-Ala-Ala-p-nitroanalide serine protease substrate (e.g., Sigma, St. Louis, Mo.) liberates p-nitroanaline, which can be measured at an absorbance of 410 nm. Elastase (e.g., Sigma) is diluted to 20 μg/ml in 100 mM tris-HCl, pH 8.0. Ten microliters AAT (at 20 mg/ml) or PBS (Control without AAT, set at 100% elastase activity) is mixed with 50 μl of diluted elastase and incubated for 20 mins at 25° C. Ten microliters of the alpha-1-antitrypsin/elastase or PBS/elastase solutions are added to 180 μl of substrate (alpha-1-antitrypsin, which was diluted to 135 μg/ml with 100 nM Tris HCl. pH 8.0) and transferred into wells of a 96 well flat bottom plate. An increase in absorbance (A) 410 nm (which indicated elastase-induced generation of p-nitroanaline) was measured serially over a 5 minute time period. Elastase alone was used as a Control (set at 100% elastase activity). The presence of a serine protease inhibitor (e.g., AAT) blocks elastase activity and suppresses liberation of p-nitroanaline (quantified as A410).

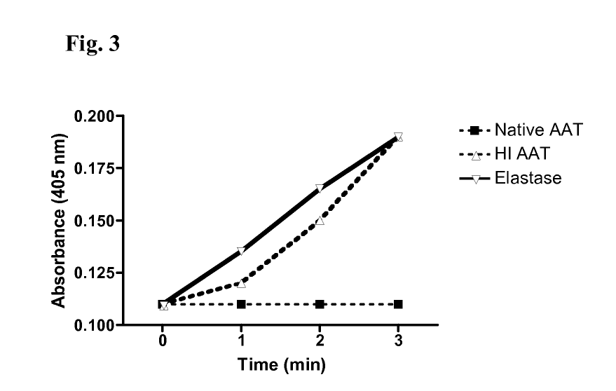

As represented in FIG. 3, elastase alone (no AAT) processed the N-Succinyl-Ala-Ala-Ala-p-nitroanalide substrate, which generated a step increase in absorbance (A410, curve labeled Elastase). Combining native (NOT heat-inactivated) AAT ablated elastase processing of the N-Succinyl-Ala-Ala-Ala-p-nitroanalide substrate and blocked the increase in A410 nm (curve labeled AAT+Elastase). In marked contrast, combining HIAAT with elastase produced a curve similar to that of elastase alone. This demonstrated that HIAAT possessed no detectable elastase neutralizing activity, since the elastase-induce generation of p-nitroanaline due to processing of the substrate N-Succinyl-Ala-Ala-Ala-p-nitroanalide was unaffected (see curve labeled HIAAT+Elastase and compare to curve labeled Elastase).

### Example 3

### Heat-Inactivated AAT (AAAT) Retains Biological Activity in Human Primary Fibroblasts

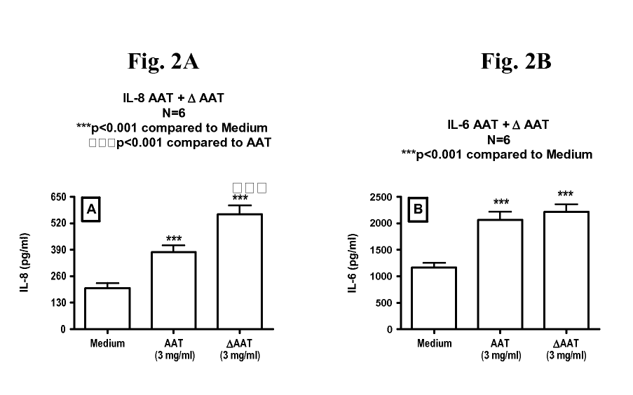

In another exemplary method, human fetal foreskin fibroblasts were obtained. Fibroblasts were grown in culture medium (e.g. RPMI 1640 medium with 10% [vol/vol] heat inactivated fetal bovine serum) in 150 mL polystyrene tissue culture flasks (Falcon, Lincoln Park, N.J.) and incubated at 37° C. and 5% CO₂ until confluent. The cells were detached using trypsin and split into 24-well polystyrene cell culture plates. The cells were then allowed to grow to confluence in these plates for 3-5 days before the actual experiments were performed. Cells were incubated (37° C., 5% CO2) in culture medium alone (Control), AAT alone, or with heat inactivated AAT (AAAT). After 24 hours of incubation (37° C., 5% CO2) supernatants were removed and frozen (−70° C.) until assay for IL-6 (Interleukin 6 FIG. 2A) and IL-8 (Interleukin 8 FIG. 2B).

FIGS. 2A and 2B represents an exemplary experiment where AAAT was shown to be devoid of serine protease inhibitor function by in vitro assay. As shown in FIGS. 2A and 2B the presence of 3.0 mg/ml Aralast AAT significantly increased the synthesis of (2A) IL 8 and (2B) IL 6 production in 24-hour fibroblast cultures (compared to control cells in medium alone, labeled Medium). Interestingly, parallel cultures conducted using 3.0 mg/ml Aralast AAAT resulted in similar production of (2A) of IL-8 and enhanced production (2B) of IL-6 in the fibroblasts compared to control (medium alone) cultures. These data demonstrate that heat inactivation of the serine protease inhibitor function of AAT does not abrogate this AAT biological activity (e.g. cytokine production). Thus, these AAT functions are separate from serine protease inhibition which accounts for certain AAT biological activities. The data illustrated in FIGS. 1, 2A and 2B demonstrate that AAT suppression of HIV in primary infected PBMC in vitro, and AAT induction of cytokines in human primary fibroblasts in vitro are both independent of AAT induced serine protease inhibition.

### Example 4

### Anti-HIV Effect of AAT

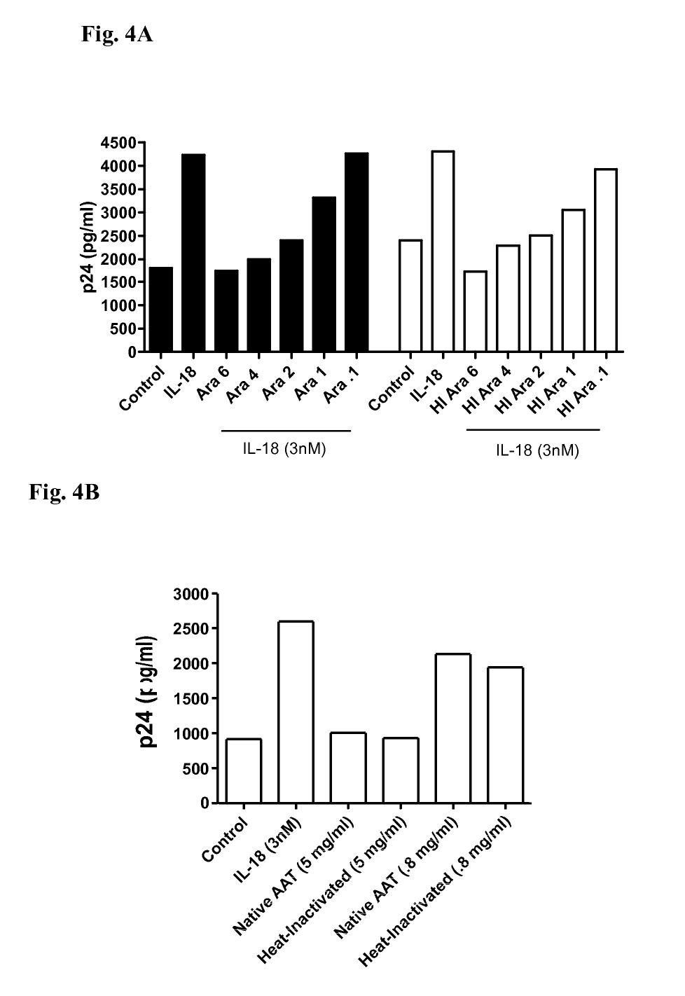

In one exemplary method it was demonstrated that AAT and HIAAT (AAAT) inhibit HIV production in chronically infected U1 cells. U1 cells are derived from the U937 human monocytic cell line by the stable incorporation of 2 copies of HIV provirus into the cell genome. These cells generate increased HIV following exposure to any of several stimuli. In these exemplary experiments, U1 cells were cultured at a density of 1×10⁶ cells per ml in 500 μl of medium consisting of RPMI 1640 medium with 10% [vol/vol] heat inactivated fetal calf serum, with penicillin 100 units/ml+streptomycin 100 μg/ml. Cells were cultured in wells of a polystyrene tissue culture plate with medium alone (control), with medium containing stimulus alone (3 nM IL 18), or with stimulus in the presence of AAT (FIG. 4A, left panel) or heat inactivated AAT (FIG. 4A, right panel). AAT was added to cultures 1.0 hr prior to the addition of IL-18 (interleukin 18) stimulus. Cultures were incubated for 24 hrs (37° C., 5% CO₂), and then lysed with 1% (vol/vol) triton X 100 and then the lysates were assayed for HIV p24 antigen using an ELISA. As shown in FIGS. 4A and 4B, IL-18 stimulated an increase in HIV production compared to medium alone (control) cultures. Stimulating U1 cell cultures with IL-18 in the presence of either unaltered (FIG. 4A, left panel) AAT or with heat inactivated AAT (FIG. 4A, right panel) resulted in dose dependent inhibition of stimulated HIV production. Comparing native with heat inactivated AAT showed very similar inhibition of p24 production. For both native and heat inactivated AAT, nearly complete HIV suppression induced by IL 18 was observed using AAT concentrations of 4 and 6 mg/ml. These results suggest very similar HIV suppression in this chronic infection model using native or heat inactivated AAT. Another experiment was performed using 0.8 or 5 mg/ml of AAT or HI AAT (FIG. 4B). For both native and heat inactivated AAT, nearly complete HIV suppression induced by IL 18 was observed using AAT of HI AAT concentrations of 5 mg/ml but not at 0.8 mg/ml. Since heat inactivation of AAT using our protocol ablates AAT serine protease inhibitory function (as documented in by an in vitro serine protease neutralization assay, data not shown), these results suggest that AAT suppression of HIV in these studies does not depend on the serine protease inhibitor function of AAT.

### Example 5

In another exemplary method, AAT (Native AAT) and HI AAT activity were analyzed for their effects on lethal toxin-induced cytotoxicity in RAW 264.7 cells (N=5). In this example, all cultures received a lethal toxin (100 ng/ml protective antigen+40 ng/ml lethal factor); p<0.001 compared to Control. This exemplary study was used to demonstrate HI AAT versus native AAT treatments on cells exposed to anthrax.

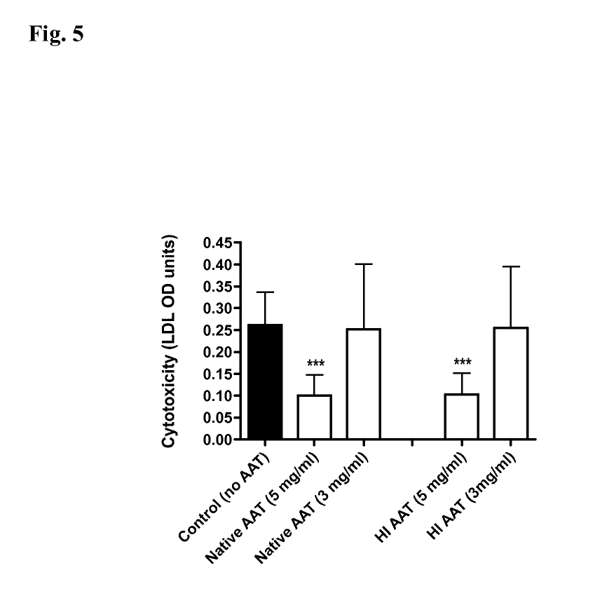

RAW 264.7 cells were cultured in medium (RPMI 1640 medium+10 heat-inactivated FBS with 100 units/ml penicillin and 100 μg/ml streptomycin) containing lethal toxin (LT) alone (control), or in medium containing LT and AAT. AAT was added 1 hr prior to addition of LT. Three hrs after addition of LT, cell culture supernatant was assayed for cytotoxicity using an LDH release assay (Promega, Madison, Wis.). As shown in FIG. 5, cells cultured in LT alone (Control, closed bar) demonstrated cytotoxicity that produced a mean of approximately 0.25 OD units (LDL OD units on the vertical axis represents increasing amounts of cytotoxicity. Five mg/ml native (not heat-inactivated) AAT significantly reduced the LT-induced cytotoxicity in the RAW 264.7 cells), whereas 3.0 mg/ml native AAT did not inhibit LT cytotoxicity. As shown in the same figure, HI AAT replicated the native AAT results almost identically, with 5.0 mg/ml HI AAT significantly reducing LT-induced cytotoxicity. In this Figure results from 5 separate experiments are shown (mean ±SEM), and *** indicates p<0.001 compared to Control (no AAT, closed bar). These data show that HI AAT is equivalent to native AAT as an inhibitor of anthrax cytotoxicity in vitro.

### Methods

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Apoptosis Assay. The protective effect of AAT on islets may address one of the major obstacles in islet transplantation today, namely the inadequacy of islet mass and post-isolation islet viability. Freshly isolated human islets activate stress signaling pathways and exhibit high rate of apoptosis due to the process of isolation, necessitating the use of more than one islet donor per diabetic patient (Nanji, (2004); Abdelli, S. et al. Intracellular stress signaling pathways activated during human islet preparation and following acute cytokine exposure. Diabetes 53, 2815-23 (2004)).

AAT dosage. Normal human plasma contains 0.8-2.4 mg/ml AAT, with a half life of 5-6 days.

### Example 6

### HI AAT Reduces LT Induced Lethality in a Murine Model of Anthrax Toxicity

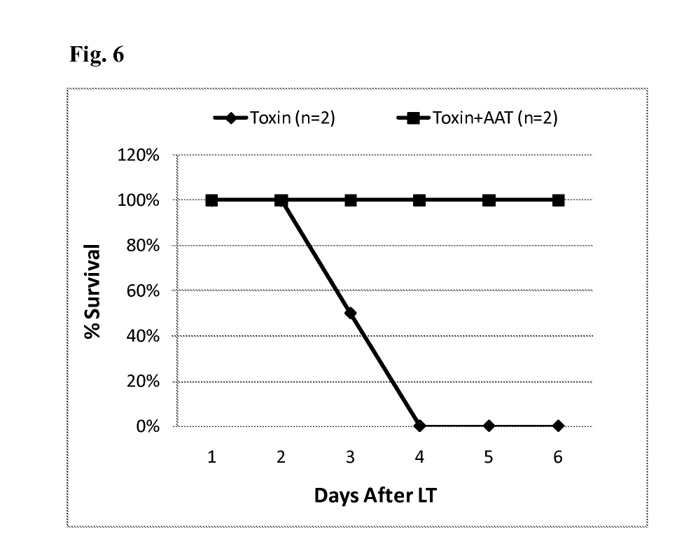

Post-Toxin Treatment: For this experiment, LT (60 μg PA and 20 μg LF) was administered as a single intraperitoneal (ip) injection to induce lethality in female Balb/c mice (Jackson Laboratory, Bar Harbor, Me.). HI AAT was introduced as a subcutaneous (sc) injection at the nape of the neck in order to separate HI AAT from LT and avoid artifact due to physical combination at the point of delivery. For the treatment arm (n=2), 2 mg of HI AAT was given immediately after LT injection (i.e., t=0). The results using this model of anthrax LT-induced lethality with HI AAT as a candidate therapy are summarized in the survival curves shown in FIG. 6. As depicted, both mice given LT alone died by day 4 (n=2; diamonds). In contrast, both of the mice given HI AAT in addition to LT remained clinically healthy through day 6 (n=2; squares). It is also important to note that the mice treated with HI AAT remained clinically healthy throughout the observation period.

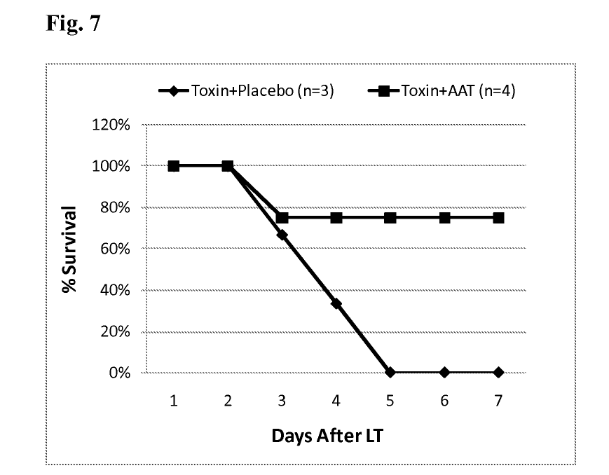

Post-Toxin Treatment with HI AAT or Placebo: For this experiment LT (60 μg PA and 20 μg LF) was administered as a single intraperitoneal (ip) injection to induce lethality in female Balb/c mice (Jackson Laboratory, Bar Harbor, Me.). HI AAT, or human albumin as a benign placebo protein, were introduced as a subcutaneous (sc) injection at the nape of the neck in order to separate HI AAT or albumin from LT and avoid artifact due to physical combination at the point of delivery. For the treatment arm, 2 mg of AAT was given once immediately after LT injection; and for the placebo arm, 2 mg of human albumin was given once immediately after LT injection. The results using this model of anthrax LT-induced lethality with HI AAT as a candidate therapy are summarized in the survival curves shown in FIG. 7. As depicted, all mice given LT and placebo (n=3) died by day 5 (diamonds). In contrast, only one of the mice given LT followed by HI AAT (n=4) died through day 7 (open squares); representing an observed treatment efficacy of 75%.

In order to assess the potential for HI AAT to ameloriate symptoms associated with exposure to anthrax toxin, the mice in this experiment were also scorred on a scale of 1 to 5 for their clinical well being over time; with a score of 1 representing completely healthy and a score of 5 indicaticatin that the mice were unable to move even after prodding. As seen in Table 1, in addition to preventing the symptom of death; with one exception, HI AAT treatment also almost entirely prevented any observable clinical symptoms associated with exposure to anthrax LT.

TABLE 1
Clinical Disease Progression in female Balb/c mice exposed
to LT and treated with HI AAT or Placebo (human albumin):

| | Toxin + Placebo (n = 3) | | | Toxin + HI AAT (n = 4) | | | |
|---|---|---|---|---|---|---|---|
| | Mouse | | | Mouse | | | |
| Day | A | B | C | D | E | F | G |
| 1 | 1 | 1 | 1 | 1 | 1 | 1 | 1 |
| 2 | na | na | na | na | na | na | na |
| 3 | dead | 4.5 | 4.5 | dead | 1 | 1 | 1 |
| 4 | dead | dead | 4.5 | dead | 1 | 1 | 1 |
| 5 | dead | dead | dead | dead | 1 | 1 | 1 |
| 6 | dead | dead | dead | dead | 1 | 1 | 1 |
| 7 | dead | dead | dead | dead | 1 | 1 | 1 |

na: no observations
1 Healthy
5 Very Sick
6 Dead

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A pharmaceutical composition comprising: alpha-1 antitrypsin (AAT) having no significant serine protease inhibition activity.

2. The pharmaceutical composition of claim 1, wherein alpha-1 antitrypsin comprises alpha-1 antitrypsin, a fragment thereof, an analog thereof, or fusion molecule thereof, having no significant serine protease inhibition activity.

3. The pharmaceutical composition of claim 1, further comprising an agent selected from the group consisting of an anti-inflammatory agent, an immunosuppressive agent, an immunomodulatory agent, an anti-microbial agent, an anti-viral agent, an anti-bacterial agent, an anti-fungal agent, an anti-parasitic agent or any combination thereof.

4. The pharmaceutical composition of claim 1, further comprising an anti-bacterial agent.

5. A method of treating a subject with a medical disorder comprising administering to the subject in need of such a treatment a therapeutically effective amount of a composition comprising, alpha-1 antitrypsin, a fragment thereof, an analog thereof, or fusion molecule thereof, having no significant serine protease inhibition activity.

6. The method of claim 5, wherein the disorder is selected from the group consisting of a viral infection, a bacterial infection, and a combination thereof.

7. The method of claim 5, wherein the disorder is selected from the group consisting of sepsis, septic shock, Acute Respiratory Distress Syndrome (ARDS), ajamian reperfusion, congestive heart failure, cardiac ischemia, stroke cerebral vascular, influenza, acute liver failure, chronic liver failure, a common cold, meningitis, Encephalitis, Candida or CMV esophagitis, pancreatitis, acute renal failure (ischemic, toxic, metabolic, thrombotic, due to collagen-vascular disease), cardiac ischemia (angina, yocardoal infarction), hepatitis (e.g. due to viruses like HAV/HBC/HCV/HSV/CMV/EBV or toxins/medications, autoimmune, ischemic), ischemia-reperfusion injury, ischemic or infectious colitis/enteritis, atypical mycobacteria, and hemorrhagic fevers (e.g. Ebola, Marbutg, Sin Nombre).

8. The method of claim 6, wherein the viral infection comprises a retroviral infection.

9. The method of claim 8, wherein the retrovirus infection is selected from the group consisting of human immunodeficiency virus (HIV) infection, AIDS (acquired immunodeficiency syndrome), influenza virus infection, hepatitis virus infection, Herpes virus infection and combinations thereof.

10. The method of claim 6, wherein the bacterial infection is selected from the group consisting of mycobacterial infection, sepsis, septic shock, bacterial meningitis, bacterial pneumonia, bacillus anthracis infection, and combinations thereof.

11. The method of claim 10, wherein the bacillus anthracis infection is derived from inhalation anthrax, cutaneous anthrax, gastrointestinal anthrax and combinations thereof.

12. The method of claim 5, wherein the composition further comprises an agent selected from the group consisting of an anti-inflammatory agent, an immunosuppressive agent, an immunomodulatory agent, an anti-viral agent, an anti-pathogenic agent, an anti-bacterial agent, a reverse transcriptase inhibitor, a protease inhibitor, and combinations thereof.

13. The method of claim 5, wherein the composition is administered orally, systemically, via an implant, intravenously, topically, intrathecally, by inhalation, nasally or a combination thereof.

14. The method of claim 5, wherein the treatment further comprises reducing or eliminating one or more symptom associated with the disorder.

15. The method of claim 5, wherein the viral infection is an influenza infection.

16. A method for reducing serine protease inhibition activity in alpha-1 antitrypsin, a fragment thereof, an analog thereof, or fusion molecule thereof comprising heating alpha-1 antitrypsin, a fragment thereof, an analog thereof, or fusion molecule thereof at a temperature of about 85° C. to about 110° C. for about 1 minute to about 1 hour.

17. The method of claim 16, further comprising assessing serine protease inhibition activity of the alpha-1 antitrypsin, a fragment thereof, an analog thereof, or fusion molecule thereof using a serine protease inhibitor activity assay.

18. A kit comprising: a composition comprising, alpha-1 antitrypsin, a fragment thereof, an analog thereof, or fusion molecule thereof, having no significant serine protease inhibition activity; and at least one container.

19. The kit of claim 18, further comprising an agent selected from the group consisting of an anti-inflammatory agent, an immunosuppressive agent, an immunomodulatory agent, an anti-microbial agent, an anti-viral agent, an anti-bacterial agent, an anti-fungal agent, an anti-parasitic agent or any combination thereof.
